(19) United States
(12) Patent Application Publication
(10) Pub. No.: US 2013/0071852 A1
(43) Pub. Date: Mar. 21, 2013

(54) Controls for Detecting Methicillin Resistant Staphylococcus Aureus (MRSA)

(71) Applicant: Life Technologies Corporation, Carlsbad, CA (US)

(72) Inventors: Erhard Ralf SCHOENBRUNNER, Moraga, CA (US); Alana BENN, Pleasanton, CA (US); Jerry BOONYARATANAKORNKIT, Davis, CA (US); Mona Danielle SHAHBAZIAN, Martinez, CA (US)

(73) Assignee: Life Technologies Corporation, Carlsbad, CA (US)

(21) Appl. No.: 13/651,112

(22) Filed: Oct. 12, 2012

Related U.S. Application Data
(63) Continuation of application No. 12/367,432, filed on Feb. 6, 2009.
(60) Provisional application No. 61/026,713, filed on Feb. 6, 2008.

(51) Int. Cl. C12Q 1/68; C12N 1/20

(52) U.S. Cl. 435/6.12; 435/252.1

(57) ABSTRACT

The invention relates to the quality control of Staphylococcus aureus testing using nucleic acid amplification-based detection assays. A Staphylococcus aureus control containing a quantified amount of the microorganism with high reproducibility across vials and which is used to calibrate, validate, or verify the performance of an MRSA detection assay and methods to test patient samples together with a control. Disclosed are specific Staphylococcus aureus strains that have a phenotype demonstrating reduced aggregation and increased consistency by Real-Time PCR compared to current Staphylococcus aureus strains used as external controls. Also disclosed is a process for increasing the reproducibility of Staphylococcus aureus strains that do not exhibit a non-aggregating phenotype.

### CROSS-REFERENCE TO RELATED APPLICATIONS

This application claims the benefit of U.S. provisional application 61/026,713 filed Feb. 6, 2008, which is fully incorporated herein by reference.

### BACKGROUND OF THE INVENTION

Staphylococcus aureus was identified as a cause of wound infections in the 1880s and was mostly fatal before the discovery of penicillin in the 1940s. Initially, penicillin was quite effective in controlling the growth of the organism in patients, but with time, resistance to 13-lactams such as penicillin appeared in some isolates of S. aureus. These isolates produced enzymes, referred to as penicillinases, that hydrolyze penicillin. Methicillin, a semi-synthetic penicillin derivative, which was resistant to penicillinases, was then introduced. However, methicillin-resistant forms of S. aureus were isolated within a year of the introduction of this new antibiotic (Hiramatsu K, Microbio. Immunol., 1995). Since that time, cases of antibiotic-resistant staphylococcus aureus known as methicillin-resistant Staphylococcus aureus (MRSA) have increased from being relatively rare in the 1960s and 1970s to being more and more widespread by the 1990s.

β-lactam antibiotics, such as penicillin and methicillin, kill gram positive bacteria by binding to enzymes known as penicillin-binding proteins (PBPs) that catalyze the formation of peptide crosslinks between glycan chains of the bacterial cell wall. Inhibition of PBPs by β-lactams results in a weakened cell wall and eventual cell lysis and death (Ghuyen J-M, Int J. Antimicrob. Agents 1997).

Methicillin-resistant Staphylococcus aureus (MRSA) is now classified as a discrete type of bacteria that is responsible for serious and deadly infections in humans. MRSA is believed to have evolved by acquiring a mobile genetic element, known as the Staphylococcal cassette chromosome (SCC) by horizontal transfer from an unknown species. The specific vector known as SCCmec cassette carries the mecA gene, which confers methicillin resistance to Staphylococci. Five types of SCCmec, designated as types I-V, are classified according to the type of recombinase they carry and their general genetic composition. The mecA gene encodes a penicillin-binding protein, PBP2a, which has a low affinity for all β-lactam antibiotics and therefore retains its ability to crosslink glycan chains in the cell wall. The resistance that this bacteria has developed over time now resists treatment with many of the antibiotics normally used to cure a staphylococcus infection, including penicillin, methicillin, and cephalosporins. When Staphylococcus is resistant to these antimicrobial agents, other similar antibiotics, such as amoxicillin and other penicillin derivatives, are also not effective. MRSA strains are particularly virulent, spread rapidly, and cause more severe infections than the typical Staphylococcus bacteria. MRSA infections are known to be nosocomial (acquired in a hospital) and are associated with open wounds, invasive devices, and the weakened immune systems which are typical of hospital patients. Accordingly, the number of MRSA infections that have been identified has increased dramatically in the last several years. In October 2007, an article in the Journal of the American Medical Association (“JAMA”) concluded that MRSA was responsible for over 90,000 serious infections and over 18,000 hospital-related deaths in the United States in 2005. Klevens, et al. JAMA, Vol. 298, No. 15 (Oct. 17, 2007).

Early detection of MRSA infection and the ability to distinguish MRSA from methicillin-sensitive Staphylococcus aureus (MSSA) is important for limiting the spread of infection, determining treatment options, and reducing healthcare cost. MRSA is often detected in a clinical setting by isolating bacteria found in the respiratory tract, wounds, catheters, blood, or nasal tracts of patients. Recently, because of the widening spread and unique danger posed by MRSA, many hospitals are beginning to routinely screen patients upon admission for the presence of MRSA. The risk for widespread infection, pneumonia, and a variety of other disorders make early detection, accurate testing, and the ability to distinguish between MSSA and MRSA infections particularly important.

Previously, the most widely used method to identify MRSA bacteria colonization in patients is the use of an ordinary culture. In culture tests, a nasal swab is collected from the nostrils of a patient and cultured, i.e. put into a special nutrient broth, or spread onto a nutrient gel, incubated, and then examined for the growth of characteristic MRSA colonies. Also, a swab may be collected from the wound site or skin lesion of a person who has been previously treated for a MRSA infection and cultured similarly. Nutrient broths and gels used to screen MRSA include salt-containing trypticase soy broth (TSB), use of mannitol-salt agar (MSA), use of MSA containing oxacillin (MSA_{Ox}), use of Mueller-Hinton agar containing oxacillin (MHA_{Ox}), and the use of MSA containing lipovitellin with an oxacillin disk (MSAL_{Ox}). Chromogenic Agar like Becton-Dickinson ChromAgar that contains enzyme substrates linked to a chromogen (color-producing compound) that are hydrolyzed by MRSA but not other organisms allows for differentiation between MSSA and MRSA. Although culture tests can be definitive, cultures are usually viewed as taking too much time, usually one to two days, such that by the time the results of a culture test are available, there is an increase risk of transmission and progression of the MRSA infection to a life-threatening state.

Faster methods of detecting MRSA have recently been developed. These new molecular test methods identify certain genetic components of MRSA, such as the mecA gene, in clinical samples. Testing for the mecA gene has only been approved recently by the FDA and Health Canada for widespread use and has the potential to detect nasal or wound carriage within hours, instead of the one to two days required by culture.

Although other methods are possible, a preferred method for detecting the presence of specific gene sequences relies on the amplification and detection of select gene sequences. These techniques are broadly classified as “nucleotide amplification technologies” (NAT). The most widespread NAT is the polymerase chain reaction or (“PCR”). In PCR, a target sequence of DNA is amplified using short oligonucleotide primers, the necessary nucleotide building blocks, and a polymerase enzyme. When the oligonucleotide primer matches and binds a target DNA sequence, the polymerase extends the primer by adding nucleotides to one end that are complementary to the target DNA sequence. The target DNA is amplified with multiple cycles of primer binding and extension.

By strategically designing the primers to detect unique DNA sequences that are present only in MRSA, PCR and other NAT based techniques are able to rapidly and specifically detect MRSA in a sample. New quantitative PCR (qPCR)-based tests use real-time polymerase chain reaction for the amplification of a MRSA-specific segment of SCCmec containing the orfX gene. In real-time PCR, an additional short oligonucleotide sequence linked to a fluorophore is used as a DNA probe for the detection of MRSA sequences during the amplification process in real time.

To assure optimum performance of the analytical assay, several types of controls are used in addition to the test samples when the assay is performed. A negative control, which lacks the analyte of interest, is designed to insure that falsely positive signals due to contamination are not observed. When the assay fails to find the target DNA in the negative control, it indicates that a positive result in the test samples is valid. A positive control, containing a known amount of the analyte of interest, is designed to insure that the assay is correctly yielding a positive result when the target sequence is present—this insures that the assay is functional. An internal control, containing a target different from the analyte of interest is designed to insure that inhibitors are not present within the sample. An external control is typically a low level control that mimics a patient sample. The purpose of an external control is to insure that all stages of the assay from extraction to detection are performing according to specifications. Because analytical assays can be sensitive yet fastidious, it is very important to include each of these complementary types of controls to evaluate the acceptability of each run of an analytical assay.

Like all assays, NAT tests such as qPCR that are used to detect MRSA require controls to verify that the assay is properly detecting the difference between positive and negative samples and that interfering compounds are not present. In practice, a testing laboratory runs an assay to test patient samples along with both positive and negative run controls to insure that the assay is not either missing positive samples (false negatives) or incorrectly identifying negative samples as positives (false positives). An external control, also known as a specimen processing control is sometimes required according to guidelines or requirements of local, state, and/or federal regulations or accreditation organizations. The two major NAT assays for MRSA include the Cepheid Xpert MRSA Assay and the BD GeneOhm MRSA Assay. The Cepheid Xpert MRSA Assay includes a specimen processing control, however the specimen is Bacillus globigii and not Staphylococcus.

In current practice, the positive control is created by simply culturing live MRSA bacteria and testing these in parallel with a sample. The use of live strains of most MRSA has several drawbacks. First, as noted above, MRSA is highly virulent, and poses a risk in ordinary handling in the laboratory. Second, these organisms tend, to aggregate in culture, which makes obtaining a consistent number of cells difficult and therefore impractical to use as a control. It is well known that bacteria self-associate in different ways, and that some tend to form into clusters of variable numbers and configurations (see FIG. 1). This self-association occurs because bacteria reproduce by replicating their DNA, growing in size, and then dividing in half. Further division can create small to large associations of cells. Bacteria are classified as “diplo” if they associate in pairs, “strepto” if they form chains, and “staphylo” if they associate in clumps or clusters. The genus Staphylococcus (Item No. 4 in FIG. 2) is so named because cells are spherical (cocci) and assemble into clusters resembling berries of a grape. This configuration is due to the fact that Staphylococci divide in two planes. Larger groupings of bacteria formed by multiple rounds of division are referred to as colonies. In Greek staphyle means bunch of grapes and coccos means granule. Under the microscope the cells appear round and form in grape like clusters. This aggregation makes it difficult to add a consistent number of Staphylococcus cells from a solution into a vial in order to produce a consistent control, standard, calibrator or reference material for diagnostic purposes.

The development and use of quality control products for NAT tests requires source material that can be dispensed in a consistent and reproducible manner. Controls for all types of MRSA testing are difficult to produce from cultures of live MRSA because aggregation of the cells leads to inconsistencies during manufacture. For the end-user, the inability to pipet a consistent amount of bacteria into an assay introduces a large amount of variability and almost negates any value of the control.

With respect to NAT assays, there are no inactive MRSA controls for NAT testing currently available. The product insert from BD GeneOhm MRSA test recommends growing cultures of MRSA to produce a positive control. The product insert directs the end-user to obtain the MRSA strain from the American Type Culture Collection (ATCC), culture the cells, and then adjust the concentration by comparing the turbidity to McFarland standards. Product inserts from the Cepheid Xpert MRSA test recommend purchasing KWIK-STIK. The KWIK-STIK MRSA positive control consists of active, lyophilized MRSA cells that are placed onto a swab and used in the assay. This control, because it contains live MRSA, presents hazards to the operator. Furthermore, this control contains high levels of bacteria. Characteristics for a safe and reliable MRSA control for NAT testing include: 1) inactivation; 2) quantified levels of bacterial cells; 3) a low level of cells (usually three times the limit of detection of the assay to insure that low amounts of MRSA can be detected); 4) close similarity to a patient sample; 5) stability; and 6) high reproducibility (minimal vial-to-vial variability).

### SUMMARY OF INVENTION

The present invention provides an MRSA control material which can serve as a non-aggregating, reliable, stable, and safe control for NAT testing. The MRSA control is comprised of strain A900159 that expresses the plasmin sensitive (pls) gene. Strain A900159 demonstrates reduced bacterial adhesion to cells and matrices and higher reproducibility in NAT tests compared to active wild-type MRSA strains that do not express the pls gene. MRSA strain A900159 can be found in the American Type Culture Collection (ATCC) repository (accession number pending). The wild-type MRSA strain of the invention shows similar reproducibility after inactivation compared to MRSA strain A900159.

This invention provides a control that can serve as a quality control for an MRSA assay, such as the BD GeneOhm MRSA assay, the Cepheid Xpert MRSA test, and other MRSA NAT tests.

This invention also provides a composition of matter comprising a Staphylococcus aureus strain stored in a liquid matrix that stabilizes Staphylococcus and mimics a patient sample. This invention provides a method for producing an MRSA control material with less than 1 log of vial-to-vial variability.

This invention also provides a method for detection of Staphylococcus aureus in a specimen by amplification of nuclear components of Staphylococcus aureus, wherein the method comprises amplification of the nuclear components of a control sample of Staphylococcus aureus, wherein NAT tests of the control Staphyloccocus aureus are consistent and reproducible due to the process and state of inactivation of the organism in the control.

In addition, this invention provides a kit for analyzing a specimen for the presence of Staphylococcus aureus, wherein the kit comprises a positive control composition comprising of Staphylococcus aureus, wherein the Staphylococcus aureus exhibits consistent and reproducible NAT assay results.

### DESCRIPTION OF FIGURES AND TABLES

FIG. 1 is a light micrograph of gram-stained Staphylococcus aureus showing aggregation of the bacteria, which makes it difficult to transfer a defined number of cells into a vial.

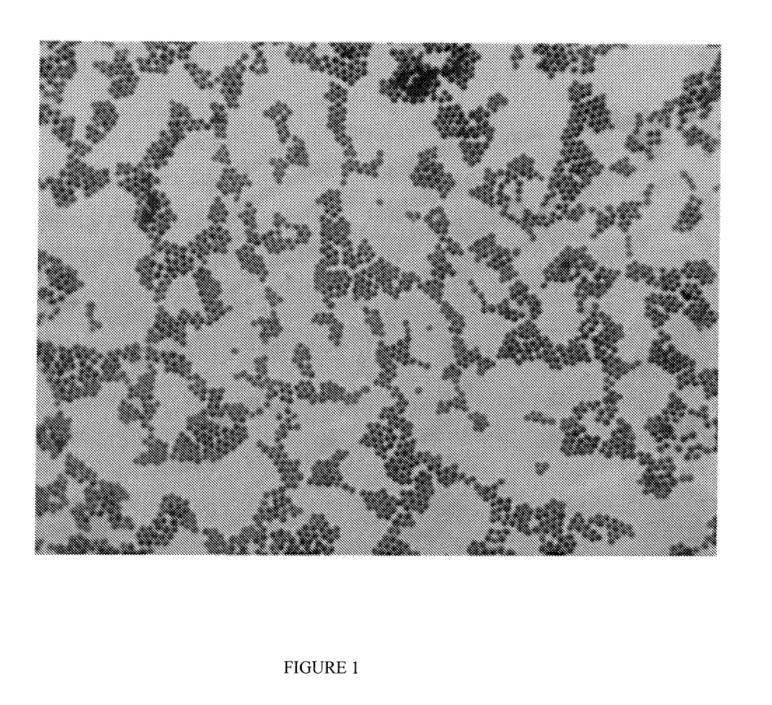

FIG. 2 shows different types of bacterial aggregation that interfere with the use of live MRSA in an assay control.

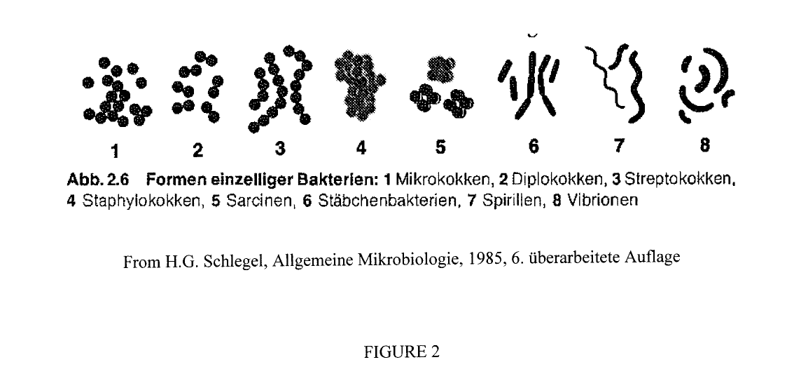

Table 1 shows the real-time quantitative Polymerase Chain Reaction (qPCR) data from the Cepheid Gene Xpert MRSA assay using active MRSA strain A900159.

Table 2 shows the Real-Time qPCR data comparing active ATCC 43300 versus inactivated ATCC 43300. The percent coefficient of variation (CV) of the active ATCC 43300 is reduced to CV near the MRSA strain A900159 in table 2 when inactivated.

Table 3 shows the Real-Time qPCRdata for MicroBiologics MRSA Kwik-Stiks.

Table 4 shows the qPCR data from two independent testing laboratories that were sent vials containing 500 CFU/ml of inactivated MRSA strain A900159.

Table 5 shows accelerated stability of a low (3-fold higher than the LOD of the Xpert MRSA Assay) MRSA control (strain A900159). The stability was tested at 25° C., 37° C., and 45° C. for up to 6 weeks.

### DETAILED DESCRIPTION OF INVENTION

In the present invention, the Staphylococcus aureus cells are a non-aggregatingmutant strain that is characterized by the expression of the plasmin sensitive (pls) gene, which encodes a cell surface protein that limits adhesion to extracellular matrices and host cells. This pls+ mutant differs from the wild-type, active Staphylococcus aureus (pls−) cells, in that the cells used for the present invention are homogenous in culture and can be grown and selected in quantities that are more readily quantifiable and reliable for purposes of formulating solutions containing a fixed quantity of cells.

This present invention provides a process to produce consistent and reproducible results in NAT-based testing methods for Staphylococcus aureus. Through the process of inactivation using cross-linking compounds, the aggregation of pls− Staphylococcus aureus cells is reduced so that the control so created has a comparable coefficient of variation to the pls+ Staphylococcus aureus strain.

The invention includes both compositions containing inactivated and non-aggregating MRSA cells, as well as methods for using such organisms. The methods of the invention include methods to calibrate, validate, or verify the performance of an MRSA detection assay and methods to test patient samples together with a control. The method thus includes performing an NAT-based assay on a clinical patient sample, to detect the presence of MRSA and testing the control of the present invention along with the clinical sample.

The non-aggregating MRSA NAT control uses a MRSA strain that expresses the plasmin-sensitive surface protein. The Pls surface protein prevents adhesion of MRSA to fibronectin and immunoglobulin G. The MRSA NAT control matrix utilizes 10 mM Tris pH 8.0, 1 mM EDTA pH 8.0, 150 mM NaCl, 2% Human Serum Albumin,15% Glycerol, 0.05% Sodium Azide, and 0.05% Gentamicin Sulfate. The MRSA NAT control is inactivated using cross-linking compounds rendering the organism non-pathogenic. Prior to inactivation, serial dilutions of the viable stock are plated in triplicate. Appropriate plates are then counted for colony growth to determine the initial titer in CFU/ml of the viable stock. After inactivation the inactive stock material is diluted to a level approximately three times the limit of detection of the Cepheid Xpert MRSA assay.

The organisms of the present invention that exhibit the non-aggregating behavior before inactivation are characterized by expression of the pls gene, are described in the following references Juuti et al., 2004 and Savolainen et al., 2001, and are available from ATCC (strain A900159, accession number pending). Generally, the organism may be characterized as any strain of MRSA that inhibits adhesion between cells. The adhesion may be inhibited by any of the methods described herein, and may be characterized by a measurement of the total number, percentage, or statistical distribution of cells exhibiting adhesion. Adhesion may be measured, accordingly, by the number of bacterial aggregates and/or the number of bacterial aggregates having a particular size or being comprised of a specified number of cells. In a preferred embodiment at least 50% of the total bacterial aggregates consist of less than 10 cells, as may be measured by discrete self-counting or by optical scanning techniques that measure a cell size distribution in solution. Inactivation may be achieved by a variety of heat, radiation, or chemical treatment techniques. Heat inactivation includes thermal or radiation treatment. Ionizing radiation includes ultraviolet light, X-ray, Electron, gamma rays, alpha particles, neutrons, or β particles. Chemical inactivation includes treatment by formaldehyde, acetaldehyde, paraformaldehyde, propionaldehyde, n-butyraldehyde, benzaldehyde, p-tolualdehyde, salicylaldehyde, phenylacetaldehyde, 2-methylpentanal, 3-methylpentanal, 4-methylpentanal, glutaraldehyde, glyoxal, malondialdehyde, succinaldehyde, adipaldehyde, phthaldehyde, maleimide, chloroacetyl, fluoracetyl, iodoacetyl, bromoacetyl, amine, hidrazide, dimethyl adipimidate (DMA), dimethyl pimelimidate (DMP), dimethyl suberimidate (DMS) and dimethyl 3,3′-dithiobisproprionimidate (DTbp). All of the inactivation techniques share the common property of terminating the virulence of the organism and altering the cell surface to reduce aggregation while preferably leaving the morphology of the cell intact to more accurately function as a control for the live organism.

In the present invention, the MRSA viable stock material is processed by adding a chemical cross-linking reagent and gently agitated. The MRSA stock material is then washed and resuspended in a protein-supplemented buffer with preservatives and a cryoprotectant. Through the process of inactivation, MRSA strains are made into MRSA NAT controls with increased consistency that is equivalent to pls+ MRSA strains.

The MRSA assay to which the control of the present invention applies is based on detection of unique nucleic acids in a Staphylococcus aureus organism. This includes community-acquired methicillin-resistant Staphylococcus aureus, hospital-acquired methicillin resistant Staphylococcus aureus, and methicillin-sensitive Staphylococcus aureus. Any nucleic acid-based amplication technology (NAT) may be used together with the controls and methods of the present invention.

The control is utilized by the end-user as a mock patient sample and preferably uses the control as specified in a product insert of the NAT assay.

The Staphylococcus aureus external controls is used to test the ability of MRSA assay systems to detect MRSA in samples resembling those of patients. The Staphylococcus aureus external control verifies the effectiveness of all steps of the assay process from extraction of the DNA to detection. An effective Staphylococcus aureus external control preferably provides a low level of organisms for detection, such as three-fold higher than the limit of detection (LOD) of the assay, so that the sensitivity limits of the assay are challenged. Additionally, the control must be manufactured in such a way to minimize vial-to-vial variability and lot-to-lot variability. Minimization of variability of the Staphylococcus aureus external control serves to validate a particular assay between runs and verifies the similar performance of different assay systems. The Staphylococcus aureus external control is stable for at least 6 months at temperatures as low as −70° C. and as high as ambient temperatures (25° C.±5° C.). The Staphylococcus aureus external control is inactivated to reduce the risk of infection to the end-user.

The control solution is preferably contained in a sealed and sterilized container and includes a preservative such as acetamide, agarose, alginate, 1-alanine, albumin, ammonium acetate, butanediol, chondroitin sulfate, chloroform, choline, dextrans, diethylene glycol, dimethyl acetamide, diinethyl formamide, dimethyl sulfoxide (DMSO), erythritol, ethanol, ethylene glycol, formamide, glucose, glycerol, a-glycerophosphate, glycerol monoacetate, glycine, hydroxyethyl starch, inositol, lactose, magnesium chloride, magnesium sulfate, maltose, mannitol, mannose, methanol, methyl acetamide, methylformamide, methyl ureas, phenol, pluronic polyols, polyethylene glycol, polyvinylpyrrolidone, proline, propylene glycol, pyridine N-oxide, ribose, serine, sodium bromide, sodium chloride, sodium iodide, sodium nitrate, sodium sulfate, sorbitol, sucrose, trehalose, triethylene glycol, trimethylamine acetate, urea, valine xylose, gentamicin sulfate, merthiolate, sodium azide, proclin.

The above preservatives stabilize the inactivated bacteria for greater than one year storage at temperatures equal to or above −20° C.±5° C.

### EXAMPLE I

### Non-Aggregating MRSA

Appropriate safety procedures were followed to prevent transmission of MRSA. MRSA strain A900159 was obtained and subcultured in trypticase soy agar. The colonies were subsequently plated on oxacillin screen agar and CHROMagar MRSA (Becton-Dickinson) to confirm the identity of the cells as MRSA. Colonies were then inoculated in trypticase soy broth and incubated at 37° C. for 16 hours. The bacterial culture was then centrifuged at 1000×g for 30 mins and the supernatant was removed. The bacterial pellet was then resuspended in a matrix containing of 10 mM Tris pH 8.0, 1 mM EDTA pH 8.0, 150 mM NaCl, 2% Human Serum Albumin, 15% Glycerol, 0.05% Sodium Azide, and 0.05% Gentamicin Sulfate. The sample was serially diluted 10,000 fold, vortexed at a medium setting and 100 μl samples were tested on the Cepheid Gene Xpert MRSA assay (Table 1). Results show that the percent CV of the titer is 24% for the strain ATCC # A900159.

### EXAMPLE II

### Inactivated MRSA Organism

A water bath was filled with distilled H₂O, then adjusted to 30° C.±5° C. An active MRSA cell suspension that was stored at −70° C. was thawed in a biosafety cabinet at ambient conditions (25° C.±5° C.). The cell suspension was then vortexed at a medium setting and mixed by reverse pipetting. The cell suspension was then pelleted by centrifuging at 14,000 RPM for 1 minute. The supernatant was then decanted and the pellet was resuspended in 1× PBS using half the initial cell suspension volume to wash the pellet. The pellet was resuspended using reverse pipetting and vortexing at a medium setting. The washed cell suspension was pelleted by centrifuging at 14,000 RPM for 1 minute. The supernatant was then decanted and the pellet was resuspended in 1× PBS using half the initial cell suspension volume to wash the pellet. The pellet was resuspended using reverse pipetting and vortexing at a medium setting. Five percent (5%) formalin solution was added at a volume equal to half the volume of the initial cell suspension and the mixture was vortexed at a medium setting for 1 minute. The cell suspension was then placed on a rocker and incubated at ambient temperature (25° C.±5° C.) for two hours. After two hours, the cell suspension was removed from the rocker or shaking device and transferred to the centrifuge. The cell suspension was spun down at 14,000 rpm for one minute and the supernatant was decanted. The cell pellet was washed twice with 1× PBS using half the volume of the initial cell suspension. After the final wash, the pellet was resuspended in the same volume as the initial volume of the cell suspension in 10 mM Tris pH 8.0, 1 mM EDTA pH 8.0, 150 mM NaCl, 2% Human Serum Albumin, 15% Glycerol, 0.05% Sodium Azide, and 0.05% Gentamicin Sulfate. The pellet was then resuspended by vortexing at a medium setting and by reverse pipetting. The MRSA cell suspension was then plated on trypticase soy agar and oxacillin screen agar to confirm inactivation.

### EXAMPLE III

### Comparison of Different Strains of MRSA to Demonstrate that the Strain Does Not Aggregate. Comparison Performed through Microbiological Staining and by Real-Time PCR

100 μl of a 10,000-fold dilution of active MRSA (ATCC# 43300) and active pls-expressing MRSA (strain A900159) were tested using the Cepheid Gene Xpert MRSA assay. Table 1 and Table 2 indicate that p/s-expressing MRSA (strain A900159) has a lower CV (24.43%) than the MRSA strain (ATCC 43300) that does not express the pls gene (141.74%). ATCC strain #43300 is recommended as the specimen processing control for the BD GeneOhm MRSA assay.

### Example IV

### Comparison of MRSA NAT Control to KWIK-STIK MRSA to Show the Concentration Differences

MRSA KWIK-STIK from MicroBioLogics was tested to determine the relative titers of the active control. The KWIK-STIK was removed from the pouch and processed as directed by the manufacturer. Briefly, the ampoule located in the cap was pinched to release the hydrating fluid. The lyophilized cell pellet was then gently mixed with the hydrating fluid to promote adherence to the swab within the KWIK-STIK. The swab was then removed and processed as a typical sample according to the Xpert MRSA Assay product insert. The titer of the MRSA KWIK-STIK is 850-fold higher than the limit of detection of the Cepheid Xpert MRSA Assay (100 CFU/ml).The MRSA KWIK-STIK is thus a high level control that does not challenge the sensitivity limits of the assay.

### EXAMPLE V

### Quantification of MRSA NAT Controls

Prior to inactivation, a viable MRSA stock was assigned a value in colony forming units per mL. Serial dilutions of 1000 fold to 100,000,000 fold of the MRSA stock material were made in 10 mM Tris pH 8.0, 1 mM EDTA pH 8.0, 150 mM NaCl, 2% Human Serum Albumin,15% Glycerol, 0.05% Sodium Azide, and 0.05% Gentamicin Sulfate. A calibrated micropipette was used to transfer 150 μl from each dilution onto three trypticase soy agar plate and the solution was spread evenly onto the plates using a sterile spreader. The plates were then inverted and incubated at 37° C.±2° C. for 16 hours. At the end of the incubation time period, all plates that had between 10 and 200 colonies were counted. To assign the value in CFU, the three replicate plates of dilutions that contained colonies numbering between 10 and 200 were visually counted. The values were adjusted by the dilution factors and plating volume and averaged across dilutions to determine the value of the MRSA stock in CFU/ml. stock. The MRSA stock concentration was calculated using the equation MRSA Stock Concentration (CFU/mL) =(Average colonies/plate x Dilution Factor)/(0.15 ml).

### EXAMPLE VI

### Stability of Inactivated MRSA NAT Controls

A MRSA low positive control, approximately 500 CFU/ml, was inactivated as described in Example II. Vials containing 135 μl of the sample were stored at ambient temperature (25° C.±5° C.) in a calibrated incubator set to 37° C.±2° C., and in a calibrated incubator set to 45° C.±2° C. Four replicates from each temperature level were tested at week 1, week 3, and week 6 (Table 5). According to the Arrhenius equation, accelerated stability for 13 weeks at ambient, 6 weeks at 37° C., and 3 weeks at 45° C. is equivalent to one year of stability at 5° C.±3° C. Data from the 37° C. and 45° C. timepoints indicates that the low positive MRSA control maintains a consistent titer over the course of the stability experiment and suggests that the control will be stable for 1 year at 5° C.±3° C.

### EXAMPLE VII

### Data Over Multiple Testing Sites to Show the Reproducibility of the Control

An inactivated MRSA control (strain A900159) targeted at a level of 500 CFU/ml, when 100 μl is used in the assay, was manufactured. Replicates were sent to two independent testing sites where the Cepheid Gene Xpert MRSA system is used. Reports from both sites indicate similar CVs at approximately 50%.

Although the foregoing has specified MRSA, the techniques are applicable to other organisms such as Bacterioidetes, Chlorobi, Bacterioidetes, Chlamydiae, Verrucomicrobia, Fibrobacteres, Acidobacteria, Thermodesulfobacteria, Deinococcus-Thermus, Thermotogae, Thermotogae, Thermomicrobia, Fusobacteria, Dictyoglomi, Aquificae, Cyanobacteria, Actinobacteria, Planctomycetes, Firmicutes, Proteobacteria, Chrysiogenetes, Nitrospirae, Deferribacteres, Choroflexi and Spirochaetes.

TABLE 1
MRSA Strain A900159

| | Relative | Relative | Avg | | |
|---|---|---|---|---|---|
| CT | log Titer | Titer | Titer | StDev Titer | CV Titer |
| 29.9 | 2.99 | 972.64 | 1252.93 | 306.11 | 24.43% |
| 29.6 | 3.08 | 1197.60 | | | |
| 29.1 | 3.23 | 1694.01 | | | |
| 29.4 | 3.14 | 1375.80 | | | |
| 30.0 | 2.96 | 907.47 | | | |
| 29.2 | 3.20 | 1580.50 | | | |
| 29.8 | 3.02 | 1042.49 | | | |

TABLE 2

| | | | | | |
|---|---|---|---|---|---|
| | Relative | Relative | Avg | | |
| CT | log Titer | Titer | Titer | StDev Titer | CV Titer |

ATCC43300 (Active)

| | | | | | |
|---|---|---|---|---|---|
| 32.1 | 2.33 | 211.50 | 1949.47 | 2763.21 | 141.74% |
| 30.2 | 2.90 | 789.93 | | | |
| 26.9 | 3.89 | 7790.52 | | | |
| 28.2 | 3.50 | 3162.28 | | | |
| 31.4 | 2.54 | 343.67 | | | |
| 30.7 | 2.75 | 558.45 | | | |
| 30.2 | 2.90 | 789.93 | | | |

ATCC 43300 (Inactivated)

| | | | | | |
|---|---|---|---|---|---|
| 31.3 | 2.57 | 368.35 | 315.01 | 89.79 | 28.50% |
| 31.3 | 2.57 | 368.35 | | | |
| 32.8 | 2.11 | 130.15 | | | |
| 31.5 | 2.51 | 320.64 | | | |
| 31.2 | 2.60 | 394.81 | | | |
| 31.4 | 2.54 | 343.67 | | | |
| 31.7 | 2.45 | 279.11 | | | |
| 31.3 | 2.57 | 368.35 | | | |

TABLE 3
Kwik-Stik MRSA

| | Relative log | Relative | Avg | | |
|---|---|---|---|---|---|
| CT | Titer | Titer | Titer | StDev Titer | CV Titer |
| 23.2 | 5.01 | 101396.76 | 81582.00 | 19842.84 | 24.32% |
| 24.1 | 4.73 | 54317.50 | | | |
| 23.4 | 4.95 | 88263.90 | | | |
| 23.5 | 4.92 | 82349.82 | | | |

TABLE 4
Inactivated MRSA Strain A900159 (500 CFU/ml)

| | | | | | |
|---|---|---|---|---|---|
| | Relative log | Relative | Avg | | |
| CT | Titer | Titer | Titer | StDev Titer | CV Titer |

Site 1

| | | | | | |
|---|---|---|---|---|---|
| 30.8 | 2.72 | 521.04 | 554.68 | 278.45 | 50.20% |
| 33.8 | 1.81 | 65.05 | | | |
| 29.8 | 3.02 | 1042.49 | | | |
| 30.9 | 2.69 | 486.12 | | | |
| 30.5 | 2.81 | 641.55 | | | |
| 31.1 | 2.63 | 423.16 | | | |
| 30.8 | 2.72 | 521.04 | | | |
| 30.3 | 2.87 | 737.00 | | | |

Site 2

| | | | | | |
|---|---|---|---|---|---|
| 30.8 | 2.72 | 521.04 | 332.78 | 167.96 | 50.47% |
| 32.0 | 2.36 | 226.68 | | | |
| 31.1 | 2.63 | 423.16 | | | |
| 32.5 | 2.20 | 160.26 | | | |

TABLE 5
Accelerated Stability Data: MRSA Positive Control

| | | Temp | Replicate | mean | StDev | Log | Titer | mean | StDev | CV |
|---|---|---|---|---|---|---|---|---|---|---|
| Name | Week | ° C. | Ct | Ct | Ct | Titer | cfu/ml | Ct | Ct | % CV |
| OptiQual MRSA | 1 | 45 | 30.10 | 29.85 | 0.57 | 2.93 | 846.66 | 1074.73 | 494.68 | 46.03% |
| OptiQual MRSA | 1 | 45 | 30.10 | | | 2.93 | 846.66 | | | |
| OptiQual MRSA | 1 | 45 | 30.20 | | | 2.90 | 789.93 | | | |
| OptiQual MRSA | 1 | 45 | 29.00 | | | 3.26 | 1815.67 | | | |
| OptiQual MRSA | 1 | 37 | 30.30 | 30.03 | 0.22 | 2.87 | 737.00 | 899.70 | 135.39 | 15.05% |
| OptiQual MRSA | 1 | 37 | 30.10 | | | 2.93 | 846.66 | | | |
| OptiQual MRSA | 1 | 37 | 29.80 | | | 3.02 | 1042.49 | | | |
| OptiQual MRSA | 1 | 37 | 29.90 | | | 2.99 | 972.64 | | | |
| OptiQual MRSA | 1 | 25 | 29.80 | 29.80 | 0.29 | 3.02 | 1042.49 | 1059.40 | 225.96 | 21.33% |
| OptiQual MRSA | 1 | 25 | 29.40 | | | 3.14 | 1375.80 | | | |
| OptiQual MRSA | 1 | 25 | 30.10 | | | 2.93 | 846.66 | | | |
| OptiQual MRSA | 1 | 25 | 29.90 | | | 2.99 | 972.64 | | | |
| OptiQual MRSA | 3 | 45 | 30.40 | 30.50 | 0.58 | 2.84 | 687.62 | 679.06 | 246.90 | 36.36% |
| OptiQual MRSA | 3 | 45 | 31.30 | | | 2.57 | 368.35 | | | |
| OptiQual MRSA | 3 | 45 | 29.90 | | | 2.99 | 972.64 | | | |
| OptiQual MRSA | 3 | 45 | 30.40 | | | 2.84 | 687.62 | | | |
| OptiQual MRSA | 3 | 37 | 31.10 | 30.53 | 0.66 | 2.63 | 423.16 | 682.77 | 322.36 | 47.21% |
| OptiQual MRSA | 3 | 37 | 29.70 | | | 3.05 | 1117.36 | | | |
| OptiQual MRSA | 3 | 37 | 31.00 | | | 2.66 | 453.55 | | | |
| OptiQual MRSA | 3 | 37 | 30.30 | | | 2.87 | 737.00 | | | |
| OptiQual MRSA | 3 | 25 | 29.50 | 30.18 | 0.67 | 3.11 | 1283.61 | 865.23 | 352.42 | 40.73% |
| OptiQual MRSA | 3 | 25 | 30.10 | | | 2.93 | 846.66 | | | |
| OptiQual MRSA | 3 | 25 | 30.00 | | | 2.96 | 907.47 | | | |
| OptiQual MRSA | 3 | 25 | 31.10 | | | 2.63 | 423.16 | | | |
| OptiQual MRSA | 3 | 5 | 30.60 | 31.00 | 0.68 | 2.78 | 598.56 | 491.08 | 216.26 | 44.04% |
| OptiQual MRSA | 3 | 5 | 31.30 | | | 2.57 | 368.35 | | | |
| OptiQual MRSA | 3 | 5 | 31.80 | | | 2.42 | 260.41 | | | |
| OptiQual MRSA | 3 | 5 | 30.30 | | | 2.87 | 737.00 | | | |
| OptiQual MRSA | 6 | 37 | 30.30 | 31.03 | 1.07 | 2.87 | 737.00 | 523.80 | 266.07 | 50.80% |
| OptiQual MRSA | 6 | 37 | 30.80 | | | 2.72 | 521.04 | | | |
| OptiQual MRSA | 6 | 37 | 32.60 | | | 2.17 | 149.52 | | | |
| OptiQual MRSA | 6 | 37 | 30.40 | | | 2.84 | 687.62 | | | |
| OptiQual MRSA | 6 | 25 | 30.70 | 30.05 | 0.70 | 2.75 | 558.45 | 957.31 | 452.08 | 47.22% |
| OptiQual MRSA | 6 | 25 | 29.30 | | | 3.17 | 1474.60 | | | |
| OptiQual MRSA | 6 | 25 | 29.60 | | | 3.08 | 1197.60 | | | |
| OptiQual MRSA | 6 | 25 | 30.60 | | | 2.78 | 598.56 | | | |

We claim:

1.-18. (canceled)

19. A method for detecting MRSA in a sample comprising securing a test sample containing bacterial DNA, amplifying a nucleotide sequence that distinguishes MRSA from MSSA and other organisms, amplifying a control solution containing inactivated MRSA or MSSA cells verifying amplification of the nucleotide sequences in the sample and control.

20. The method of claim 19 wherein the control solution comprises at least 50% of the bacterial aggregates contain less than 10 cells.

21. The method of claim 19 wherein control solution comprises a cross-linking agent.

22. The method of claim 21, wherein the cross-linking agent is selected from a group consisting of formaldehyde, acetaldehyde, paraformaldehyde, propionaldehyde, n-butyraldehyde, benzaldehyde, p-n itrobenzaldehyde, p-tolualdehyde, salicylaldehyde, phenylacetaldehyde, 2-methylpentanal, 3-methylpentanal and 4-methylpentanal.

23. The method of claim 20, wherein the control solution comprises a cross-linking agent that comprises two or more reactive functional groups.

24. The method of claim 23, wherein the cross-linking agent is a dialdehyde.

25. The method of claim 24, wherein the dialdehyde is selected from the group consisting of glutaraldehyde, glyoxal, malondialdehyde, succinaldehyde, adipaldehyde and phthaldehyde and combinations thereof.

26. The method of claim 21, wherein the cross-linking agent comprises at least one functional group from the group consisting of NHS imidate, maleimide, chloroacetyl, fluoroacetyl, iodoacetyl, bromoacetyl, amine, and hydrazide and combinations thereof.

27. The method of claim 20, wherein the cross-linking agent is a imidoester.

28. The method of claim 27, wherein the imidoester is selected from the group consisting of dimethyl adipimidate (DMA), dimethyl pimelimidate (DMP), dimethyl suberimidate (DMS) and dimethyl 3,3′-dithiobisproprionimidate (DTBP) and combinations thereof.

29. The method of claim 19 wherein the Staphylococcus aureus methicillin resistant.

30. The method of claim 19 wherein the Staphylococcus aureus methicillin sensitive.

31. A method to test an assay that detects the presence of MRSA in a sample comprising: performing Staphylococcus aureus detection assays on a plurality of MRSA and MSSA controls, wherein the plurality of MRSA controls contain discrete, predetermined quantities of inactivated, non-aggregating Staphylococcus aureus.

32. The method of claim 31 wherein the controls are in a solution comprising a cross-linking agent.

33. The method of claim 32, wherein the cross-linking agent is selected from a group consisting of formaldehyde, acetaldehyde, paraformaldehyde, propionaldehyde, nbutyraldehyde, benzaldehyde, p-n itrobenzaldehyde, p-tolualdehyde, salicylaldehyde, phenylacetaldehyde, 2-methylpentanal, 3-methylpentanal and 4-methylpentanal and combinations thereof.

34. The method of claim 31, wherein the cross-linking agent comprises two or more reactive functional groups.

35. The method of claim 34, wherein the cross-linking agent is a dialdehyde.

36. The method of claim 35, wherein the dialdehyde is selected from the group consisting of glutaraldehyde, glyoxal, malondialdehyde, succinaldehyde, adipaldehyde and phthaldehyde and combinations thereof.

37. The method of claim 31, wherein the cross-linking agent comprises at least one functional group from the group consisting of NHS imidate, maleimide, chloroacetyl, fluoroacetyl, iodoacetyl, bromoacetyl, amine, and hydrazide and combinations thereof.

38. The method of claim 31, wherein the cross-linking agent is a imidoester.

39. The method of claim 38, wherein the imidoester is selected from the group consisting of dimethyl adipimidate (DMA), dimethyl pimelimidate (DMP), dimethyl suberimidate (DMS) and dimethyl3,3′-dithiobisproprionimidate (DTBP) and combinations thereof.

40. The method of claim 31 wherein the Staphylococcus aureus methicillin resistant.

41. The method of claim 31 wherein the Staphylococcus aureus methicillin sensitive.

42.-52. (canceled)
